(19) United States
(12) Patent Application Publication
(10) Pub. No.: US 2025/0325582 A1
(43) Pub. Date: Oct. 23, 2025

(54) A hydrogen peroxide-based therapeutic spray for protection and treatment of mucosa

(71) Applicant: Vijay Duggal, Elmhurst, NY (US)

(72) Inventor: Vijay Duggal, Elmhurst, NY (US)

(21) Appl. No.: 18/703,350

(22) PCT Filed: Mar. 1, 2023
(86) PCT No.: PCT/US2023/014252

§ 371 Date: Aug. 3, 2025

Related U.S. Application Data
(60) Provisional application No. 63/315,975, filed on Mar. 2, 2022.

(51) Int. Cl. A61K 33/40; A61K 9/00; A61K 47/02; A61K 47/10; A61K 47/22; A61K 47/24

(52) U.S. Cl. CPC A61K 33/40; A61K 9/0043; A61K 47/02; A61K 47/10; A61K 47/22; A61K 47/24

(57) ABSTRACT

The present invention is about a nasal spray or mucous membrane spray system for protection and treatment of mucosa, particularly nasal mucosa, from infections and allergies as well as its protection from oxidative damage caused by the use of a hydrogen peroxide-based nasal spray. A hydrogen peroxide-based spray works by redox reaction between the hydrogen peroxide and mucus or bodily fluids of mucosa; however it results in damage to mucosa in the long-term. The present invention mitigates this damage while providing therapeutic relief to the user suspected of being exposed to an infection or an allergy. The present invention enables cleansing, disinfection and treatment of nasal cavities by using a hydrogen peroxide-based nasal spray which loosens mucus from nasal cavities by oxidation of mucus therein and simultaneously enables enhanced delivery of one or more pharmaceutically acceptable compounds into nasal mucosa by the energy of redox reaction. The present invention minimizes the discomfort associated with the administration of hydrogen peroxide by enabling gradual oxidation of mucus or bodily fluids between 10-180 seconds. It protects mucosa form the harsh effects of hydrogen peroxides with skin moisturizers and disclosed therapeutic compounds which are penetrated into mucosa by the energy of redox reactions. The present invention enables treatment of localized mucosa as well as enhanced delivery of medicaments by the energy of redox reaction for use in the treatment of various medical conditions in less than 5 minutes.

A directional spray bottle equipped with a metered pump is configured to deliver a pharmaceutical composition having 1.1-1,000 centipoise viscosity comprising hydrogen peroxide and one or more therapeutic compound in the form of aerosolized droplets <100 microns in sizes from a pharmaceutical composition consisting essentially of hydrogen peroxide 0.5%-3% w/w, a therapeutic compound and deionized water. When administered over mucosa, the aerosolized droplets form a substantially self-adhering reactive coating (2′) (FIG. 1a) therein <0.25 millimeter thick, thereby oxidizing mucus or bodily fluids therein between 10-180 seconds, thereby loosening mucus or bodily fluids from the mucosa, and simultaneously enables delivery of the therapeutic compound(s) and deionized water into mucosa by the energy of redox reaction between the hydrogen peroxide and mucus or bodily fluids.

### CROSS REFERENCE TO PROVISIONAL FILING

This application takes benefit of U.S. provisional application No. 63/315,975 filed on Mar. 2, 2022.

### TECHNICAL FIELD

The present invention is about a therapeutic spray for protection and treatment of mucosa, particularly nasal mucosa, from infections and allergies as well as its protection from oxidative damage caused by the use of a hydrogen peroxide-based nasal spray. A hydrogen peroxide-based spray works by redox reaction between the hydrogen peroxide and mucus or bodily fluids of mucosa; however it results in damage to mucosa in the long-term. The present invention mitigates this damage while providing therapeutic relief to the user suspected of being exposed to an infection or an allergy. The present invention enables cleansing, disinfection and treatment of nasal cavities by using a hydrogen peroxide-based nasal spray which loosens mucus from nasal cavities by oxidation of mucus therein and simultaneously enables enhanced delivery of one or more pharmaceutically acceptable compounds into nasal mucosa by the energy of redox reaction. The present invention minimizes the discomfort associated with the administration of hydrogen peroxide by enabling gradual oxidation of mucus or bodily fluids between 10-180 seconds. It protects mucosa form the harsh effects of hydrogen peroxide with skin moisturizers and disclosed therapeutic compounds which are penetrated into mucosa by the energy of redox reactions. The present invention enables treatment of localized mucosa as well as enhanced delivery of medicaments by the energy of redox reaction for use in the treatment of various medical conditions in less than 3 minutes.

A directional spray bottle equipped with a metered pump is configured to deliver a pharmaceutical composition having 1.1-1,000 centipoise viscosity comprising hydrogen peroxide and one or more therapeutic compounds in the form of aerosolized droplets <100 microns in sizes from a pharmaceutical composition consisting essentially of hydrogen peroxide 0.5%-3% w/w, a therapeutic compound and deionized water. When administered over mucosa, the aerosolized droplets form a substantially self-adhering reactive coating (2′) (FIG. 1a) therein <0.5 millimeter thick, thereby oxidizing mucus or bodily fluids therein between 10-180 seconds, thereby loosening mucus or bodily fluids from the mucosa, and simultaneously enables delivery of the therapeutic compound(s) and deionized water into mucosa by the energy of redox reaction between the hydrogen peroxide and mucus or bodily fluids in less than 3 minutes.

One embodiment of the present invention is about a pharmaceutical composition comprising hydrogen peroxide for use in a method of disinfecting nasal mucosa while protecting said nasal mucosa from oxidative damage by the hydrogen peroxide. One embodiment of the present invention is about a nasal spray system for disinfecting nasal mucosa while protecting the nasal mucosa from oxidative damage due to a redox reaction therein and for simultaneously providing therapeutic relief to a subject suspected of being exposed to an infection or allergy. One embodiment of the present invention is about a method of disinfecting mucosa of a subject suspected of being exposed to an infection or allergy by an energy of redox reaction, while protecting said mucosa against damage caused by the redox reaction, and simultaneously providing therapeutic relief.

### BACKGROUND ART

Cleansing and disinfection of nasal cavities and respiratory passages is essential for prevention and treatment of many allergies and viral infections so is delivery of therapeutic compounds and antimicrobial drugs into the mucous membranes. The conventional methods of enabling cleansing and disinfection of nasal cavities are nasal washes of saline solution by using a neti pot such as disclosed by prior arts: US20130041329A1 and US20130039950A1. Such methods are cumbersome as they require an apparatus, plenty of water and a sink. Viral infections must be treated as soon as they are suspected. An individual who may be away from home may not have the convenience of utilizing such methods. Many pharmaceutically acceptable antimicrobial and therapeutic compounds are known but their direct application into nasal cavities does not allow sufficient penetration into the mucous membranes as they are often covered with mucus or bodily fluids.

It is known that viral infections get trapped in the mucus within the nasal cavities, but how to remove the infected mucus is not known. The focus of the prior arts has been to kill viruses in situ which often cause harm to the mucosa. The present invention has taken a different approach to viral infections: It discloses a nasal spray to remove the infected mucus from the nose while simultaneously delivering pharmaceutical compounds into the mucous membranes thereby enabling disinfection and treatment in about 3 minutes. The present invention uses aerosolized droplets <100 micron is sizes comprising hydrogen peroxide as the active agent which loosens mucus from nasal cavities for removal and exposes the surface of mucosa for enabling enhanced delivery of the disclosed pharmaceutical compounds into mucosa. The inventor has personally demonstrated one embodiment of the invention as a nasal spray in a video clip posted at: www.gunkex.com/video1

Several hydrogen peroxide based compositions are known such as disclosed by patents U.S. Pat. Nos. 4,927,627A, 4,900,721A, 6,348,187, US20220061320A1, EP2680818B1, AU2005244462B2, U.S. Pat. No. 8,637,085B2, CA2564763C, CA1277899C, U.S. Pat. Nos. 5,665,332A, 7,060,253B1, 5,665,332A and 7,060,253B1 but none of them discloses a nasal spray or a mucous membrane spray or method of drug delivery for cleansing, disinfection and treatment of nasal cavities or mucous membranes. Despite the urgent need during the pandemic, no government agency, medical body or manufacturer has come up with a system or a method to remove the infected mucus from deep within the nasal cavities while simultaneously enabling delivery of antimicrobial compounds into the mucous membranes.

The closest prior art may be the applicant's own previous application US20220061320A1 (patent granted) which is limited only to specific compositions. It needs to be noted that the provisional application for present invention was filed prior to the publication of US20220061320A1 on Mar. 3, 2022, therefore it may not be considered a prior art against the applicant.

Hydrogen peroxide as liquid is known to be harmful to mucous membranes of nasal cavities, therefore its use as such is not recommended by the US Food and Drug administration. The administration of 3% hydrogen peroxide solution to rinse nasal cavities by using conventional methods such as neti pot is cumbersome, harmful, and even fatal. (https://youtu.be/5Oowl-JuFlo). New Jersey government website www.NJ.gov lists hydrogen peroxide as a hazardous substance and provides several public warnings against its use for nasal or throat applications: https://tinyurl.com/2p8jcyra

No data is available to confirm the safety of hydrogen peroxide on nasal mucosa for long-term use. The present invention has confirmed that not only hydrogen peroxide nasal spray by itself is harmful to the nasal mucosa after a few days of use, it is discomfortable to use, and offers no therapeutic relief to the user. FIG. 0x illustrates an example of oxidation (redox) damage caused to a mucosa by hydrogen peroxide if used by itself.

The present invention discloses means of protecting mucosa from oxidative damage due to topical administration of hydrogen peroxide on mucosa, particularly nasal mucosa while providing therapeutic relief through mucosa. Despite the urgent need during the pandemic no major manufacturers have come up with hydrogen peroxide based nasal sprays, and the Food and Drug Administration (FDA) has made no recommendation of its use as such. The present invention has taken several inventive steps to overcome the aforementioned problems and proved that when mixed with the disclosed therapeutic compounds and administered as fine mist aerosol, it can be safely utilized. The inventor of the present invention has personally demonstrated the unexpected results in a video clip posted at: www.gunkex.com/video1

### SUMMARY OF INVENTION

### Technical Problem

To protect nasal mucosa from oxidative damage caused by topical administration of hydrogen peroxide on mucosa, particularly nasal mucosa, and enable cleansing, disinfection and treatment of nasal cavities by enhanced delivery of one or more pharmaceutically acceptable compounds into mucosa by the energy of redox reaction in less than 3 minutes for treatment of a medical condition.

### Solution to Problem

While hydrogen peroxide is considered a hazardous substance, it is discomfortable to use and is harmful to the mucous membranes, the present invention has found its utility for cleansing, disinfection and treatment of nasal cavities and mucous membranes and for enabling enhanced drug delivery through mucous membranes for treatment of various medical conditions while mitigating its harmful effects. It discloses hydrogen peroxide for use in a nasal spray or a mucous membrane spray device, in a composition or in a method for treatment of nasal disinfection, loosening mucus for removal from nasal cavities, for enabling nasal irrigation, or for dispersion of one or more pharmaceutically acceptable compounds into the mucus membranes for treatment of a medical condition. The present invention discloses a number of means and methods to protect nasal mucosa from the damaging effects of redox reaction between hydrogen peroxide and mucus or bodily fluids while providing therapeutic relief to a subject suspected of being exposed to an infection or allergy. Among the means include droplet sizes of 100 microns or less, viscosity of the pharmaceutical composition, the use of skin conditioners and disclosed therapeutic compounds which contribute to mitigate this damage, mitigate discomfort as well as provide therapeutic relief.

The nasal mucosa is often covered with mucus which prevents efficient penetration of topical drugs when administered as nasal sprays. The present invention has discloses a composition, a method and device which enables to loosen mucus by oxidation by hydrogen peroxide in about 10-180 seconds and simultaneously enables enhanced delivery of the drugs or therapeutic compounds into the mucous membranes by the energy of redox reaction between the hydrogen peroxide and mucus or bodily fluids. The loosened mucus can be expelled by the user or can be wiped off. This system and method enables cleansing, disinfection and treatment, as well has enhanced delivery of drugs through mucosa to treat various medical conditions in less than 3 minutes.

A fine mist spray bottle is configured to produce aerosolized droplets <100 microns in sizes, from a pharmaceutical composition comprising hydrogen peroxide as the active delivery agent and at least one pharmaceutically acceptable deliverable compound. When administered into nasal cavities or over mucous membranes the aerosolized droplets oxidize mucus or bodily fluids therein, thereby loosening mucus or bodily fluids from mucous membranes, and simultaneously disperse the deliverable compound(s) into the mucous membranes by the energy of redox reaction in less than 3 minutes. This process is used not only for enabling cleansing, disinfection and treatment of local mucous membranes but also for delivery of medicaments into the body to treat various medical conditions.

It is an object of the present invention to protect mucosa from oxidative damage by hydrogen peroxide and mitigate pain and discomfort associated with the administration of hydrogen peroxide. The present invention has discovered that the amount of hydrogen peroxide that comes into contact with the mucous membranes for a given time is directly proportion to the pain and discomfort it causes. The present invention has discovered that the harmful and discomforting effects of hydrogen peroxide on nasal mucosa, can be mitigated when administered as aerosolized droplets <100 microns in sizes along with the disclosed skin moisturizers, pharmaceutical polymers and other therapeutic agents disclosed hereinafter. Increasing the viscosity of the liquid solution with the skin moisturizers and polymers minimizes the amount of hydrogen peroxide that comes into contact with the mucous membranes at a given time thereby enabling gradual oxidation of mucus mitigating pain and discomfort while preventing damage to nasal mucosa.

The present enables gradual redox reaction, by means of predetermined viscosity of the disclosed pharmaceutical compositions. Utilizing a predetermined viscosity 1.1-1,000 centipoise enables a gradual oxidation/reduction reaction in which the transfer of electrons occurs at a controlled rate between hydrogen peroxide and bodily fluids; the energy released is spread out over a longer period of time, say 10-180 seconds. This allows for a more controlled release of energy over time, thereby resulting in mitigation of pain and controlled delivery of the disclosed compounds into mucosa prolonging their therapeutic effect while protecting mucosa from damage caused by sudden release of energy. The protection against the damaging effects of hydrogen peroxide is further enhanced when the disclosed skin moisturizers such as glycerin, aloe vera gel and pharmaceutical polymers are used as the means to increase viscosity and enable mucoadhesion for the formation of substantially self-adhering reactive coating 2′ (FIG. 1a).

In one embodiment of the invention the spray bottle utilized for the present invention is comprised of metered pump configured to deliver about 0.05 ml-0.2 ml per pump of the liquid solution. The objective is to inject only a predetermined amount of hydrogen peroxide per pump into the nasal cavities to minimize discomfort. For young healthy adults about 0.15 milligram of hydrogen peroxide per pump into each nasal cavity is generally found to be suitable. The word ‘about’ refers to 20% (±) variation. For both cavities this amount would be about 0.3 milligram per pump. Generally 2-3 pumps of sprays with a gap of about 10-30 seconds are advised to dull the pain and discomfort, therefore making the dosage of hydrogen peroxide per application as 0.6-1.2 milligram. For children this dosage is preferred to be half, while for domesticated animals this dose is preferred to be double. The aforementioned dosage is for nasal application. For mucous membranes of other body parts this dosage is intended to be double. The aforementioned dosages are exemplary, they do not limit the scope of the present invention.

The present invention has discovered that only a thin self-adhering reactive coating (2′) (FIG. 1a) of hydrogen peroxide <0.5 millimeter thickness formed the aerosolized droplets <100 microns in sizes is sufficient to loosen mucous and bodily fluids from nasal cavities and mucous membranes and for enabling dispersion of the disclosed pharmaceutical compounds into the mucous membranes. The present invention has further discovered that sudden oxidation of mucus within the nasal cavities by hydrogen peroxide, for example less than 10 seconds, is discomfortable while controlled gradual oxidation, for example 10-180 seconds, is reasonably acceptable by most young healthy adults.

To achieve controlled or gradual oxidation of mucus with the nasal cavities the present invention utilizes glycerin, aloe vera gel, mucoadhesive polymers, surfactants, emulsifiers and thickening agents which enable adherence of the aerosolized droplets with the mucous membranes for 10-180 seconds without substantial dripping enabling in order to form the reactive coating (2′) <0.5 millimeter thickness. In one embodiment of the invention the viscosity of the liquid solution 1.1- 1,000 centipoise is utilized as means to minimize dripping and for enabling controlled gradual redox reactions. The thickness of the reactive coating (2′) and the size of droplets used vary depending upon the mucoadhesive polymers, surfactants, emulsifiers, alcohol, astringents or thickening agents used. For example the thickness of the reactive coating (2′) based primarily on hydrogen peroxide, monoterpenoids, alcohol and astringents can be 0.1 millimeter thick, and droplet sizes can be 1-30 microns. When aloe vera gel, mucoadhesives polymers and/or thickening agents are used to increase viscosity, the droplet sizes can be 20-100 microns (VMD) to ensure smooth flow of sprays and the reactive coating (2′) thickness between 0.1-1 millimeter thick. Thickness of 0.5 millimeter as claimed is an average thickness of the coating; it will vary between about 0.01-1 millimeter. The aforementioned measurement are provided only as a reference, they are not intended to limit the scope of the present invention as these measurements are depended upon several other factors such as the number of times a spray is applied, dryness of nasal cavities of an individual and environmental factors such as temperature and humidity. As the droplets move with gravity when administered they may gather within the nooks and crannies of the mucous membranes to make the reactive coating (2′) thicker at certain places as well. The coating measurements provided herein are average when applied. These measurements begin to change as soon as the reactive coating (2′) is applied due to the redox reactions. Also as the discomfortable levels are subjective from one individual to another, and the present invention is equally applicable for domesticated animals, the time duration measurements provided herein are for examples only. The droplet sizes of <100 microns, viscosity of the liquid solution 1.1-1000 centipoise, methods of mucoadhesion such as electrostatic deposition, diffusion, adsorption and/or hydrogen bonding of the fine mist droplets with the mucous membranes by using the disclosed surfactants, alcohol, astringents, solubilizing agents, mucoadhesives, polymers and thickening agents all contribute to the formation of self-adhering reactive coating (2′) <0.5 millimeter thickness which enables gradual redox reactions between hydrogen peroxide and mucus or bodily fluids.

To mask the discomfort associated with the administration of hydrogen peroxide into the nasal or oral cavities the present invention utilizes aromatic and flavoring compounds which also provide therapeutic relief. To mitigate harmful effects of hydrogen peroxide on mucous membranes the present invention utilizes skin moisturizers and humectants such as glycerin (glycerol) or derivatives there of, aloe vera gel and pharmaceutically acceptable polymers. In one embodiment of the invention the present invention utilizes therapeutic compounds such as backing soda and citric acid to enable gradual controlled oxidation by hydrogen peroxide droplets while providing therapeutic relief. The use of aromatic or flavoring compounds is omitted for domesticated animals.

It an object of the present invention to treat localized medical conditions associated with the mucous membranes as well as to enable enhanced drug delivery of pharmaceutical compounds through mucous membranes to treat various medical conditions such as drug delivery into the Central Nervous System (CNS). In one embodiment of the invention the aerosolized droplets <100 micron in sizes comprising hydrogen peroxide are utilized to deliver therapeutic salts, topical steroids, antihistamines, non-steroidal anti-inflammatory drugs (NSAIDs), analgesic compounds, antibiotic compounds and local anesthetic compounds through the mucosa in the form of nasal spray or throat spray. In one embodiment of the invention the medicaments are specific to gum sprays, ear sprays or for the treatment of urological tracts delivered by the energy of redox reaction. Many of these drugs are identified by a chemical structure comprising one or more five-membered rings, six-members heterocyclic rings or a combination thereof. The present invention has discovered that hydrogen peroxide as an active agent is compatible to deliver the compounds identified by the aforementioned chemical structures particularly when the compound also comprises oxygen, nitrogen, sulfur or phosphorus or a combination thereof within the chemical structure.

The administration of aerosolized droplets <100 microns in sizes comprising hydrogen peroxide over the mucus membranes results in the formation of miniature clusters of active microscopic oxygen bubbles 1′ (FIG. 1b) which act as the vehicles to disperse the disclosed pharmaceutical compounds into the mucous membranes by mechanical action. These vehicles move powered by the energy of redox reactions enabling deeper penetration of the disclosed compounds into the mucous membranes.

The disclosed nasal spray and oral spray device provides convenient and expeditious means for cleansing, disinfection and treatment of nasal cavities and mucous membranes without the need of a bulky equipment thereby minimizing viral infection risks as soon as they are suspected. This device and method provides preventive measures against viral infections, a first-aid treatment system to viral infections as well as delivery of medicament into the mucous membranes to treat various medical conditions. The method comprises the steps of:
(1) Spraying aerosolized droplets <100 microns in sizes comprising hydrogen peroxide 0.25%-3% w/w; one or more pharmaceutically acceptable deliverable compound(s) and deionized water into nasal cavities or over mucous membranes
(2) Forming a reactive coating (2′) <0.5 millimeter thickness of said aerosolized droplets within the nasal cavities or over the mucous membranes
(3) Allowing the aerosolized droplets to react with mucus within the nasal cavities or mucous membranes for 10-180 seconds.

As a result, the aerosolized droplets oxidize mucus within the nasal cavities, loosen mucus for removal and simultaneously disperse the pharmaceutically acceptable deliverable compound(s) into the mucous membranes by the energy of redox reaction(s) in less than 3 minutes.

The term ‘self-adhering reactive coating (2′)’ refers to a property of aerosolized droplets in which at at least 50% of the administered droplets adhere to the interior surfaces of nasal cavities for at 10 seconds without dripping out. This is enabled by glycerin, aloe vera gel, mucoadhesives, polymers surfactants or astringents as disclosed herein. Glycerin, aloe vera gel, mucoadhesives and pharmaceutical polymers are used to achieve 1.1-1,000 centipoise viscosity which enable gradual oxidation of mucus by the between 10-180 seconds minimizing discomfort; this is in contrast to a sudden oxidation if hydrogen peroxide by itself were to to used as a nasal spray causing a greater discomfort and damage to nasal mucosa.

It is object of the present invention to minimize decomposition of hydrogen peroxide and promote economical utilization of substances used for the disclosed nasal sprays or mucous membrane sprays. To achieve these objectives the present invention discloses retail packaging options for the nasal spray or mucous membrane sprays as illustrated in FIGS. 2a, 2b and described hereinafter.

It is an object of the present invention to enable targeted drug delivery thereby minimizing harmful effects of antimicrobial agents. For example one embodiment of the present invention is a dental spray comprising 2%-3% hydrogen peroxide, chlorhexidine gluconate 0.01-0.2% and an aromatic compound or flavor. This dental spray enables to target only a specific gum of the user requiring treatment utilizing only one or two sprays using 0.1 to 0.2 ml of the liquid solution. This is in contrast to achieving the same results by a mouthwash utilizing about 5 ml of the same solution and exposing the entire oral cavity to the harmful effects of chlorhexidine gluconate. Furthermore the chlorhexidine gluconate is delivery further deeper into the mucous membranes by the energy of redox reaction.

### Advantageous Effects of Invention

The aerosolized nasal sprays or mucous membrane sprays of the present invention can be conveniently administered into the nasal cavities or over mucous membranes to enable cleansing, disinfection and treatment in less than three minutes without the need for bulky equipment such as a neti pot or sink. A nasal spray bottle in the context of the present invention is essentially a device to remove the infected mucous from the nasal cavities and provide therapeutic relief in less than 3 minutes. The disclosed system and method enables to get the infected mucus out from the nose to guard against viral infections while delivering therapeutic compounds into the mucous membranes. The disclosed mucous membrane sprays provide therapeutic relief and enable enhanced drug delivery though mucous membranes by the energy of redox reaction to treat various medical conditions. The utilization of the present invention is equally applicable to the domesticated animals.

### BRIEF DESCRIPTION OF DRAWINGS

FIG. 0x illustrates an example of oxidation damaged caused to a mucosa by hydrogen peroxide.

FIG. 1a illustrates a schematic section though a mucous membrane. It shows the formation of self-adhering reactive coating 2′ by the aerosolized droplets when administered over the mucous membrane. This results in the formation of miniature clusters of active microscopic oxygen bubbles (1′). The active oxygen bubbles 1′ move by the energy generated during the oxidation/reaction reactions between the hydrogen peroxide and mucus and bodily fluids of the mucous membranes by mechanical transport (FIG. 1b). Mechanical transport refers to the movement of active microscopic oxygen bubbles (1′) over the mucous membrane illustrated in FIG. 1b. The movement is driven by the energy generated during the redox reactions between the hydrogen peroxide and mucus and bodily fluids of the mucous membranes. The active oxygen bubbles (1′) facilitate the drug delivery of the deliverable compounds by the mechanical movement. This movement is a result of the energy released during the redox reactions between the hydrogen peroxide and the mucous membrane fluids, which create an energetic driving force for the bubbles to move. Furthermore, this also enables active transport (ATP) of the deliverable compounds into the cell membrane proteins by the movement of the active microscopic oxygen bubbles (1′) by the energy of redox reaction(s), further enhancing the delivery of the compounds. A non-limiting example of the movement of the active microscopic oxygen bubbles (1′) can be seen in the video clip posted at: www.gunkex.com/video1.

FIG. 1b is a snapshot of microscopic view of the miniature clusters of active microscopic oxygen bubbles (1′).

FIGS. 2a, 2b illustrate options for retail packaging of the nasal spray and mucous membrane sprays for cleansing, disinfection and treatment of nasal cavities or mucous membranes.

FIGS. 3a, 3b illustrate examples of heterocyclic compounds.

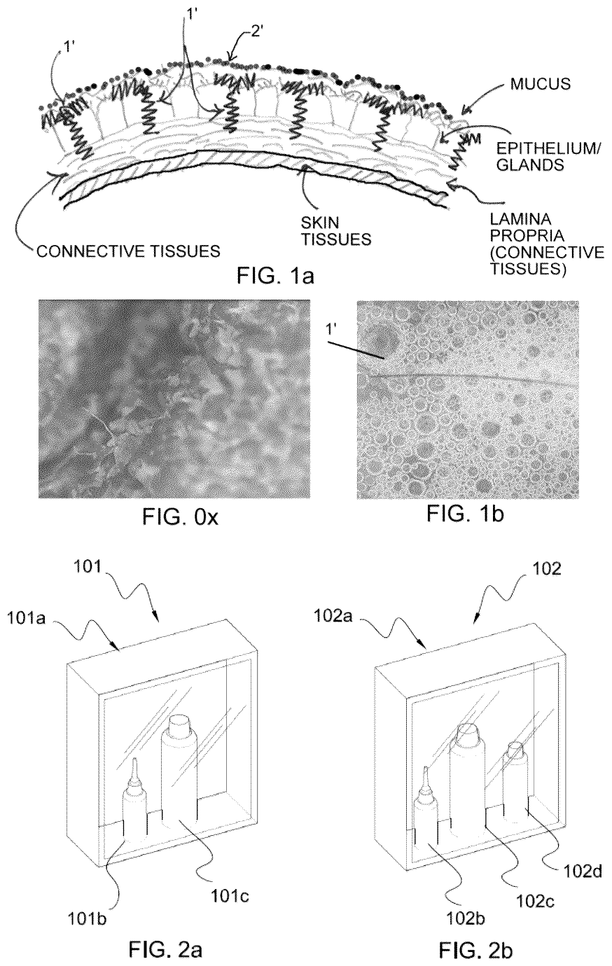

### DESCRIPTION OF EMBODIMENTS

The present invention utilizes hydrogen peroxide in different ways for achieving nasal disinfection, nasal irrigation and drug delivery of various therapeutic compounds and medicaments into the mucous membranes by the energy of redox reaction(s) produced during the oxidation of mucus, enzymes and impurities present within the bodily fluids of mucous membranes. It discloses means several means and methods to protect nasal mucosa from the harmful effects caused by the redox reaction while providing therapeutic relief to a subject suspected of being exposed to an infection or allergy. The present invention has discovered that only a thin coating (2′) of <0.5 millimeter thickness formed by hydrogen peroxide fine mist of droplets <100 micron in sizes consisting of hydrogen peroxide 0.5%-3% w/w and deionized water delivered by using a directional spray bottle is sufficient to enable loosening of mucus from nasal cavities for removal and simultaneously enabling dispersion of the hydrogen peroxide droplets and deionized water into the mucous membranes for nasal disinfection and irrigation while mitigating the harmful effects of the redox reaction. In different embodiments of the invention additional therapeutic compounds are added to the aqueous solution to further enhance the therapeutic value, user experience and treatment of various deceases. The unexpected results achieved by the present invention are demonstrated by the inventor in a video clip posted at: www.gunkex.com/video1

In one embodiment, the present invention is about a pharmaceutical composition consisting essentially of hydrogen peroxide and glycerin or a humectant for use as a medicament for protecting mucosa from oxidative damage when administered as aerosolized droplets <100 microns in sizes for treatment of a medical condition. In one embodiment, the present invention is about an aerosolized composition consisting essentially of hydrogen peroxide, glycerin or a skin moisturizer, a therapeutic compound, and deionized water for use in a method for protecting nasal mucosa from oxidative damage while enabling treatment of a medical condition. One embodiment of the invention is: Hydrogen peroxide for use in a composition for dispersion of one or more pharmaceutically acceptable compound(s) into mucous membranes. One embodiment of the invention is: Hydrogen peroxide for use in a method of enabling relief from nasal congestion. In one embodiment of the invention hydrogen peroxide is for use in a nasal spray or a mucous membrane spray device, in a composition or in a method for treatment of nasal disinfection, loosening mucus for removal from nasal cavities, for enabling nasal irrigation, or for dispersion of one or more pharmaceutically acceptable compounds into the mucus membranes for treatment of a medical condition. A fine mist spray bottle is configured to produce aerosolized droplets <100 microns in sizes, from a pharmaceutical composition comprising reactive oxygen species (ROS) such as hydrogen peroxide as the active delivery agent and at least one pharmaceutically acceptable deliverable compound. When administered into nasal cavities or over mucous membranes the aerosolized droplets oxidize mucus or bodily fluids therein, thereby loosening mucus or bodily fluids from mucous membranes, and simultaneously disperse the deliverable compound(s) into the mucous membranes by the energy of redox reaction in less than 3 minutes.

The present invention discloses a nasal spray or a mucous membrane spray for cleansing, disinfection and treatment of nasal cavities or mucous membranes and for the delivery of various pharmaceutical compounds into mucous membranes in less than 3 minutes. The system consists essentially of a fine mist spray bottle configured to produce aerosolized droplets <100 micron in sizes, a pharmaceutically acceptable peroxide having oxidation state of oxygen as O^{-1;} one or more pharmaceutically acceptable deliverable compounds; and deionized water. When administered as a nasal spray or mucous membrane spray the aerosolized droplets form a reactive coating (2′) <0.5 millimeter thickness over the mucous membranes oxidizing mucus or enzymes therein for 10-180 seconds, loosening mucus or bodily fluids from the mucous membranes, and thereby enabling dispersion of the deliverable compounds into the mucous membranes by the energy of redox reaction(s). During the redox reactions microscopic oxygen bubbles 1′ (FIGS. 1a, 1b) are formed which enable the dispersion of the deliverable compound(s) and deionized water into the mucus membranes.

In one embodiment of the invention a fine mist spray bottle is configured to produce a directional spray of aerosolized droplets <100 microns in sizes from a liquid solution, consisting of hydrogen peroxide 0.5%-3% w/w and deionized water. When the aerosolized droplets are administered into nasal cavities, they form a reactive coating (2′) therein <0.5 millimeter thickness, loosen mucus for removal from said nasal cavities by oxidation of mucus between 10-180 seconds, and simultaneously enable dispersion of the deionized water into mucous membranes by the energy of redox reaction thereby enabling disinfection and irrigation of the nasal cavities in less than three minutes. To further minimize discomfort and enhance therapeutic value and treatment option, different additional substances are added as disclosed hereinafter.

In one embodiment of the invention the nasal spray or mucous membrane system consists essentially of a fine mist directional spray bottle configured to produce aerosolized droplets <100 microns in sizes from a liquid solution having viscosity about 1.1-1,000 cP (centipoise), consisting essentially of one or more pharmaceutically acceptable peroxides 0.25%-5% w/w; one of more aromatic compounds 0.005%-5% w/w; one or more skin moisturizers, humectants or mucoadhesive polymers, emulsifiers, solvents or surfactants 0.1%-30% w/w; and deionized water. When administered, the aerosolized droplets are substantially adhering to the interior walls of nasal cavities or mucus membranes without substantial dripping for at least 10 seconds forming a reactive coating (2′) therein <0.5 millimeter thickness. The aerosolized droplets oxidize mucus or bodily fluids therein therein for 10-180 seconds, loosen mucous or bodily fluids from the mucous membranes, and simultaneously disperse the aromatic compounds, skin moisturizers, humectants and mucoadhesive polymers into the mucous membranes enabling cleansing, disinfection and treatment. In one embodiment of the invention the liquid solution further consists essentially of benzalkonium chloride, benzoic acid, boric acid, chlorhexidine gluconate, cetylpyridinium chloride (CPC) or a pharmaceutically acceptable cationic surfactant 0.01%-1% w/w. In one embodiment of the invention the liquid solution further consists essentially of sodium bicarbonate, magnesium sulfate, aluminum sulfate, sodium borate, sodium benzoate, sodium phosphate, sodium salicylate, sodium carbonate peroxide, sodium percarbonate, sodium citrate or sodium carboxymethylcellulose 0.01%-1% w/w. In one embodiment of the invention the liquid solution further consists essentially of an antiseptic, a topical steroid, an antihistamine, a nonsteroidal anti-inflammatory drug (NSAID), an analgesic compound, an antibiotic compound, an antipruritic compound, a local anesthetic compound or a central nervous system (CNC) agent 0.01%-5% w/w. In one embodiment of the invention the liquid solution further consists essentially of alcohol from 5%-50% v/v. All formulations comprise pharmaceutically acceptable preservatives, stabilizers chelating agents or buffering agents to ensure a predetermined shelf life of the formulations.

In one embodiment of the invention is a nasal spray, comprising a nasal spray bottle configured to produce aerosolized droplets <100 microns in sizes from a liquid solution comprising hydrogen peroxide 0.25%-3% w/w; one of more aromatic or flavoring compounds 0.005%-5% w/w; one or more skin moisturizers, humectants, mucoadhesive polymers, emulsifiers, solvents or surfactants 0.1%-30% w/w; and deionized water. The aerosolized droplets when administered into nasal cavities form a thin coating (2′) average <1000 microns thick over mucous membranes therein oxidizing mucus for 10-180 seconds, loosen mucous for removal, and simultaneously disperse the aromatic or flavoring compounds, skin moisturizers, humectants, mucoadhesive polymers, emulsifiers, solvents or surfactants into the mucous membranes. The dispersion of the deliverable compounds is enabled by the energy or redox reactions and the formation of miniature clusters of active microscopic oxygen bubbles 1′ (FIGS. 1a, 1b) over the mucous membranes during the oxidation process. In one embodiment of the liquid solution further comprises an effective amount of an antiseptic, a topical steroid, an antihistamine, a nonsteroidal anti-inflammatory drug (NSAID), an analgesic compound, an antibiotic compound, an antipruritic compound, a local anesthetic compound or a central nervous system agent (CNC).

One embodiment of the invention is a nasal spray or mucus membrane spray, comprising a fine mist spray comprising aerosolized droplets <100 microns in sizes, comprising hydrogen peroxide from 0.25% to 3% w/w; one or more aromatic compounds from 0.005%-2.5% w/w; glycerin, a derivative of glycerol, aloe vera gel or a mucoadhesive polymer from about 0.1% to 30%; and deionized water. In one embodiment of the invention the aerosolized droplets further comprise ethanol 5%-50% v/v.

One embodiment of the invention is a nasal spray, comprising a fine mist spray comprising aerosolized droplets <100 microns in sizes, comprising hydrogen peroxide from 0.25% to 5% w/w; chlorhexidine gluconate 0.01%-1%; one or more aromatic or flavoring compounds from 0.005%-5% w/w; and deionized water. When administered the aerosolized droplets oxidize and loosen mucous or bodily fluids over the mucous membranes and simultaneously disperse the chlorhexidine gluconate and the aromatic or flavoring compounds into the mucous membranes by the energy of redox reactions.

One embodiment of the invention is a mucous membrane spray, comprising a fine mist spray comprising aerosolized droplets <100 microns in sizes, comprising hydrogen peroxide from 0.25% to 5% w/w; borax 0.1%-5%; glycerin, a derivative of glycerol, or a mucoadhesive polymer from about 0.1% to 30%; and deionized water. When administered the aerosolized droplets oxidize and loosen mucous or bodily fluids over the mucous membranes and simultaneously disperse the borax, the glycerin, the derivative of glycerol, or the mucoadhesive polymer into the mucous membranes by the energy of redox reactions.

In one embodiment of the invention the method of cleansing, disinfection and treatment of nasal cavities or mucous membranes comprises the steps of:

(1) Spraying aerosolized droplets <100 microns in sizes comprising hydrogen peroxide 0.25%-3% w/w; one or more pharmaceutically acceptable deliverable compound(s) from group 1, and deionized water into nasal cavities or over mucous membranes. The group 1 being comprising:
at least one aromatic or flavoring compound 0.005%-5% w/w;
at least one skin moisturizer or a humectant 0.1%-30% w/w;
at least one pharmaceutically acceptable mucoadhesive polymer, a surfactant, an emulsifier
or a thickening agent 0.1%-10% w/w;
   (2) Forming a reactive coating (2′) of said aerosolized droplets within the nasal cavities or over the mucous membranes and allowing the aerosolized droplets to react with mucus within the nasal cavities or mucous membranes for at least 10 seconds. The aerosolized droplets oxidize mucus, loosen mucus for removal and simultaneously disperse the pharmaceutically acceptable deliverable compound(s) from group 1 into said mucous membranes in less than 3 minutes.

The present invention primarily utilizes stabilized hydrogen peroxide or food grade hydrogen peroxide 0.25%-5% w/w as the active delivery agent. The utilization of other pharmaceutical acceptable peroxides or oxidizing agents such as urea-hydrogen peroxide, benzoyl peroxide, sodium percarbonate is within the scope of the present invention. In one embodiment of the invention the aerosolized droplets comprises a combination of hydrogen peroxide and urea-hydrogen peroxide or benzoyl peroxide as the active delivery agents. The active delivery agents are used to deliver one or more pharmaceutically acceptable deliverable compounds. The oxidation state of the active delivery agents when administered as aerosolized droplets is O⁻¹; this is to state that the peroxides are not decomposed when mixed with the deliverable agents as liquids, and are active to carry out the intended oxidation of mucus and bodily fluids. The peroxides and/or hydrogen peroxide utilized for the present generally comprises chemical stabilizers, buffers, chelating compounds and catalytic inhibitors such as: Sodium phosphate, sodium phytate, phosphoric acid, phosphorous acid, acetanilide, sodium stannate, derivatives of tin and/or edetate disodium (EDTA).

In one embodiment of the invention the disclosed pharmaceutical compositions are pre-filled in a fine mist spray bottle while in another embodiment the pharmaceutical compositions are contained in a separate container enabling the user to fill or refill the fine mist spray bottle as needed. In one embodiment of the invention to extend the shelf life of hydrogen peroxide and the pharmaceutically acceptable deliverable compounds they are packaged in separate containers; the user can combine these ingredients as and when needed. The aforementioned spray bottles and liquid containers can be packaged into as single retail package or they can be packaged in separate retail packages. The aforementioned configurations enables economical utilization of the constituents of the system of treatment of nasal cavities, respiratory passages and mucous membranes. FIGS. 2a, 2b illustrate embodiments of the present invention in the form of nasal cleansing and disinfection systems 101 and 102. System 101 comprises retail package 101a which is comprised of a nasal spray bottle 101b and liquid container 101c. Nasal spray bottle 101b can be pre-filled with the liquid solution comprising hydrogen peroxide and pharmaceutically acceptable deliverable compounds or it can be empty enabling the user to fill in the aforementioned liquid solution from container 101c as and when needed. System 102 comprises retail package 102a which is comprised of nasal spray bottle 102b, a liquid container 100b and liquid container 102d. Liquid container 102c is filled with stabilized hydrogen peroxide 0.210%-10% w/w or a combination of pharmaceutically acceptable peroxides 0.210%-10% w/w and deionized water. Liquid container 102d is filled with one or more pharmaceutically acceptable deliverable compounds. The method of utilizing system 102 comprises mixing formulations from containers 102c and 102d by predetermined proportions and filling the mixture into nasal spray bottle 102b as and when needed.

One of the challenges with the compositions comprising peroxides is maintaining their integrity when mixed with the deliverable agents as peroxides tend to oxidize most substances and get decomposed in the process. The present invention has discovered that pharmaceutical compounds comprising five-membered or six-membered heterocyclic rings within their chemical structure are generally compatible with the hydrogen peroxide to enable their enhanced delivery into the mucous membranes. The compounds utilized for the present invention generally do not comprise transition metals (groups 3-12) as they are found to decompose peroxides. The present invention utilizes chemical buffers, inhibitors or chelating agents to maintain the integrity of hydrogen peroxide even when they are not specifically claimed as such. The following are non-limiting examples of pharmaceutically acceptable therapeutic compounds and medicaments. As a reference to the claims they are categorized into groups; these groups however do not represent a scientific or chemical classification. Several substances listed in these groups serve multiple purses, hence they should be viewed as making he claims indefinite when more than one groups are listed within a single claim. The compounds that are incorrectly defined or categorized in the present application can be omitted without affecting the scope of the present invention:

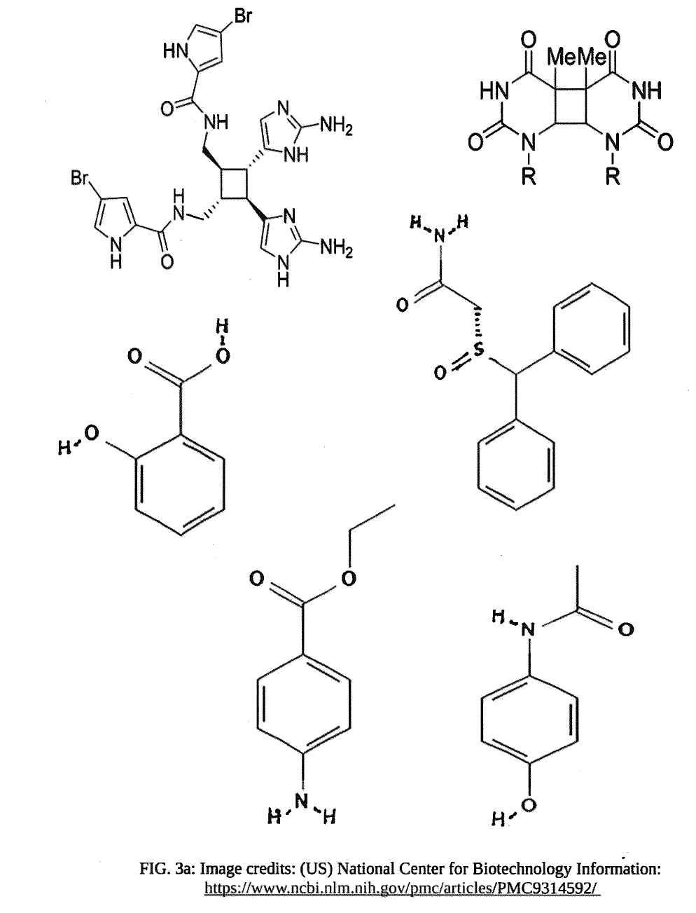

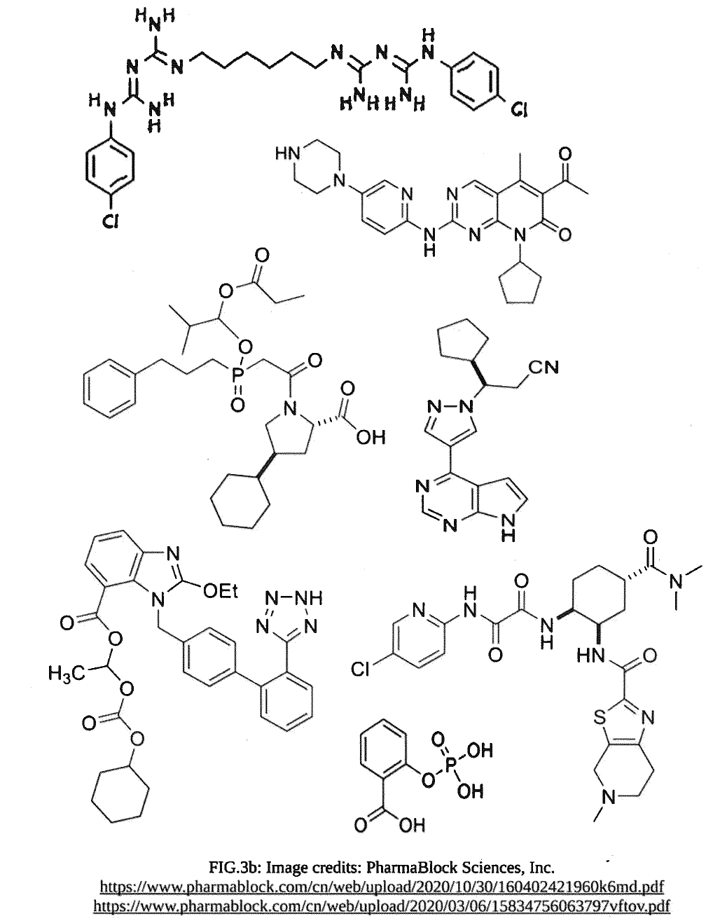

Group (a): Pharmaceutically acceptable heterocyclic compounds. Some examples of such compounds are shown in FIGS. 3a and 3b. In one embodiment of the present invention the heterocyclic compounds are identified by their chemical structure comprising one or more five-membered heterocyclic rings, one or more six-membered heterocyclic rings, or a combination thereof. The heterocyclic compounds utilized for the present invention comprise at least one hetero atom of nitrogen (N), oxygen (O), sulfur(S) or phosphorous (P) within their chemical structure. Some examples are furan, indole and/or purine serving as scaffolds and building blocks for many pharmaceuticals. Many heterocyclic derivatives and substituted heterocyclic groups combining various hetero atoms are known in the art such as as identified by patents: US20060293320A1, U.S. Pat. Nos. 8,729,077B2, 8,394,968B2 and 8,143,414B2. Some examples are aromatic compounds, monoterpenoids, alcohols, phenol and its derivatives. Medicaments and substances such as acetaminophen, aspirin, benzocaine, hydroxyzine, chloroxylenol, cloflucarban, bacitracin, salicylic acid, chlorhexidine gluconate, niacinamide, cannabis, caffeine and niccotine. Central Nervous System (CNS) stimulants such as armodafinil and modafinil. Nonsteroidal anti-inflammatory drugs (NSAIDs), ibuprofens, antipruritics, analgesic compounds, antihistamines, antibiotics, antiseptics, local anesthetics, steroids, antifungal agents such as azoles and azoles derivatives, nucleic acids, vitamins and vaccines comprising the aforementioned chemical structures or substructures.

Group (b): Pharmaceutically acceptable aromatic compounds, monoterpenes, terpenoids 0.005%-5% w/w such as: Aromatic and therapeutic substances found in essential oils. Some examples are: Phenolic monoterpenoids such as thymol, carvacrol, eugenol 0.005%-1% w/w. Effective amount of methyl salicylate, eucalyptol, geraniol, menthol, raspberry ketone, p-cymene, cinnamaldehyde, salicylic acid, nootkatone, terpineol, α-terpineol, limonene, borneol, terpinen-4-ol, camphor, cannabidiol (CBD), cresol, phenol or derivatives of the aforementioned compounds. While some substances such as camphor do not meet the chemical classification of being aromatic, in the context of the present invention such substances are considered aromatic as is being part of a monoterpene. Pharmaceutically acceptable flavoring compounds 0.1%-10% w/w such as geranyl acetate, methyl formate, ethyl acetate, jasmone, benzaldehyde, vanillin, anethole and benzenediol, germinane and germine. Synthetic fragrances. The aromatic compounds are dissolved in a pharmaceutically acceptable solvent, ethanol, dimethylsulfoxide (DMSO), a solubilizing agent, emulsifier and/or surfactant for even distribution by the disclosed aerosolized droplets. Some examples include sugar alcohols such as sorbitol mannitol, erythritol, maltitol and xylitol. Aromatic compounds and flavors identified in the US Food and Drug Administration (FDA) Inactive Ingredients Database (July 2022): https://tinyurl.com/57yxvxjw.

Group (c): Pharmaceutically acceptable skin moisturizers, or humectants 0.1%-30% w/w such as: Aloe vera gel, glycerin (glycerol) or derivatives thereof such as polyglycerol, monoglycerides and diglycerides. Pharmaceutically acceptable polymers or polycols such as a hydrophilic polymers, polyethylene glycol (PEG) or its derivatives (PPGs) such as PEG-40, propylene glycol (PG) or its derivatives and copolymers. Hydroxy acids such as α-hydroxy acids (AHAs, BHAs); hydrophilic silicone polymers and silica aerogels. Hyaluronic acid, salicylic acid, sodium lactate, sodium PCA or lactic acid. Urea and its derivatives such as hyaluronic acid urea, allantoin (10-ureidohydantoin) and imidazlidynyl urea. Dimethicone. Vitamin A, vitamin B3, D-Panthenol, pantothenic acid and vitamin E. Peptides and amino acids. Hydrogenated castor oil, lanonil oil, hydrogenated lanolin, paraffin(s), paraffin oil. Ceramides and collagen. Glycolic acid, ascorbic acid, citric acid. Pharmaceutically acceptable polymers 0.1%-30% w/w. Excipients for skin care such as emollients and occlusives. https://www.ncbi.nlm.nih.gov/pmc/articles/PMC410010343/

Group (d): Pharmaceutically acceptable polymers 0.1%-30% w/w such as: Mucoadhesives, gelling agents, thickening agents or polycols. Polyethylene glycol (PEG) or its derivatives (PPGs) such as PEG-40. Derivatives of cellulose with carboxymethyl groups (—CH2—COOH), carboxymethylcellulose (CMC), cellulose gum, Xanthan gum, Emulthix™, Disodium EDTA, carbomer, sodium carboxymethylcellulose (SCMC), hydrogels, polysaccharide polymers and their derivatives, acrylates, carrageenan, gellan, polyacrylic acid, glycerin (glycerol) or derivatives thereof, aloe vera gel, hyaluronic acid, poly (acrylic acid) polymers, poly (hydroxyethyl methylacrylate), poly (ethylene oxide), poly (vinyl pyrrolidone), poly (vinyl alcohol), chitosan and its derivatives. Poloxamers, poloxamer 407. Hydrophilic polymers. Mucoactive agents such as mucolytics. https://tinyurl.com/2fwptvpz

Group (e): Alcohols such as: Ethanol, isopropyl alcohol, cetyl alcohol, aromatic alcohols, sugar alcohols, benzyl alcohol.

Group (f): Pharmaceutically acceptable surfactants, emulsifiers, solublizing agents or astringents 0.01%-1% such as: Nonionic, anionic, cationic or amphoteric surfactants such as: Hexadecyltrimethylammonium (‘cetrimide’), chlorhexidine and its derivatives such as chlorhexidine gluconate, chlorhexidine acetate, xylenol, chloroxylenol, benzalkonium chloride, hexadecylpyridinium chloride, cetylpyridinium chloride (CPC), polysorbate-20, polysorbate-80, polyoxyethylene, glycoside, cremophor RH 40, and derivatives of quaternary ammonium compounds comprising oxygen. Bile acids. Lipids and phospholipids; palmitic acid, stearic acid, oleic acid, linoleic acid. Self-emulsifying drug delivery systems (SEDDS). Alum and derivatives thereof, witch-hazels, dimethylsulfoxide (DMSO). Mucoactive agents such as mucolytics.

Group (g): Pharmaceutically acceptable therapeutic salts such as: Organic salts. Pharmaceutically acceptable salts of sodium such as sodium bicarbonate, sodium borate, sodium benzoate, sodium fluoride, sodium phosphate, sodium salicylate, sodium carbonate peroxide, sodium percarbonate, sodium citrate, sodium carboxymethylcellulose and sodium phytate. Alum and its derivatives. Magnesium sulphate, zinc gluconate, carboxylate salts and benzethonium chloride. https://en.wikipedia.org/wiki/Oxyanion|https://tinyurl.com/2p8kpvny

Different combinations of substances from Groups (a)-(g) can be combined to formulate the therapeutic compounds and medicaments as pharmaceutical compositions or emulsions which as used to generate the aerosolized compositions by using a directional spray bottle of the present invention. The pharmaceutical compositions can be in the form of micro-emulsions or nanoemulsions comprising one or more constituents from the aforementioned groups. 645

A combination of means such as viscosity 1.1-1,000, droplets sizes <100 microns, the use of mucoadhesives and polymers that also serve as skin moisturizers, and the use of surfactants and alcohol enable the formation of substantially self-adhering reactive coating <0.25 microns thick which enables gradual oxidation of mucus between 10-180 seconds, enables loosening said mucus or bodily fluids from mucosa, and simultaneously enables delivery of the disclosed pharmaceutical compounds into mucosa by an energy of redox reaction between said hydrogen peroxide and bodily fluids in less than 3 minutes.

The term ‘consisting essentially of’ in the context of the present invention excludes all substances of prior arts that do not play an active role as disclosed by the present invention. The term ‘consisting essentially of’ in the context of the present invention however includes substances like preservatives, stabilizers and chemical buffers that prolong the shelf life of the disclosed pharmaceutical compositions. The term ‘miniature clusters of active microscopic oxygen bubbles’ is used as a means to describe random clusters of active oxygen bubbles in any formation. The term ‘sufficient amount’ refers to pharmaceutically acceptable amount to provide therapeutic relief to treat a medical condition, or the amount suitable as a preventive measure or treatment. The systems and methods disclosed herein are applicable for treatment of mucosa of all body parts such as throat, mouth, respiratory passages, gums, ears and genitals. In one embodiment of the invention the aforementioned systems and methods are used to treat hair follicles, open wounds, disinfection of body parts during surgical procedures, damaged skin etc. The droplet sizes provided in microns refer to Volume Mean Diameter (VMD). The viscosity measurements are at room temperature 21° C.

The present application includes several exemplary hyperlinks that provide additional information. However as these external hyperlinks are subjected to be being changed, they are provided only as a reference. They are not part of this application. The embodiments of the present invention as described herein are for examples only; they should not be used in a limiting sense to interpret the scope of the present invention. Different embodiments of the present invention can be combined to developed other embodiments. Any embodiments developed by extrapolation or combination of the compositions, methods of drug delivery, devices and systems disclosed herein are within the scope of the present invention.

Group (h): Pharmaceutically acceptable oxygen-containing thickening agents, mucoadhesive polymers, galling agents and/or mucus altering agents such as: Derivatives of cellulose with carboxymethyl groups (—CH2—COOH), cellulose gum, carboxymethylcellulose (CMC), Xanthan gum, Emulthix™, carbomer, sodium carboxymethylcellulose (SCMC), polyisobutylene, pectin, gelatin, lectin-based polymers, thiolated polymers, hydrophilic polymers, hydrogels, polysaccharide polymers and their derivatives, acrylates, carrageenan, gellan, polyacrylic acid, glycerin, aloe vera gel, vegetable gums, ethylene glycol-bis (Baminoethyl ether), tetraacetic acid (EGTA). https://tinyurl.com/5evr5tae|https://tinyurl.com/vfjxatsb|https://tinyurl.com/2p9b4br5

Group (i): Pharmaceutically acceptable hydrogen peroxide stabilizers, buffers and catalytic inhibitors such as disclosed in patents: U.S. Pat. Nos. 2,426,154A, 7,045,493B2, 7,156,178B2, 2,426,154A, 7,045,493B2 and 7,156,178B2. Some examples are: Sodium phosphate, phosphorous acid, acetanilide, sodium stannate, derivatives of tin, chelating agents such as edetate disodium (EDTA).

Group (j): Oxygen-containing medications such as: Nonsteroidal anti-inflammatory drugs (NSAIDs), ibuprofens, antipruritics, analgesic, antihistamines, antibiotics, local anesthetics, topical steroids, nucleic acids, vitamins and vaccines such as: Diphenhydramine (benadryl), hydroxyzine, corticosteroids, fluticasone, tropolone, proguanil and its derivatives, lidocaine, pramoxine, benzocaine, nalfurafine, centrally-acting κ-opioid, oclacitinib, jewelweed, selective serotonin reuptake inhibitors (SSRIs), niacinamide, ubiquinone, antimicrobial peptides, acetanilide phenacetin, cholinergic agents, acetaminophen, oxymetazoline HCL, antioxidants, peptides, retinol, vitamins, FluMist Quadrivalent, Covaxin, etc. https://en.wikipedia.org/wiki/Cholinergic https://en.wikipedia.org/wiki/Antipruritic

Group (k): Pharmaceutically acceptable compounds comprising ions of sodium, ions of chloride, ions of organic salts or a combination thereof such as sodium chloride; compounds comprising formates, acetates, benzoates, carboxylates, phenolates, oxalates, citrates, gluconates and sorbates. https://en. wikipedia.org/wiki/List of organic salts

Group (l): Water, evaporated water, deionized water and/or oxygenated water

Key principles/claims of the present invention:

1. A device for treatment of nasal cavities or mucous membranes comprising:
a spray bottle or nebulizer configured to produce aerosolized fine mist of reactive oxygen species (ROS) from a liquid solution consisting essentially of hydrogen peroxide from about 0.1% to 5% w/w, w/v; one or more pharmaceutically acceptable mucosal antimicrobial or therapeutic agent(s) from about 0.01% to 5% w/w, w/v; and water;
said liquid solution being having viscosity from 0.8-10,000 cps for enabling at least 50% of said fine mist to adhere to said nasal cavities or said mucous membranes without dripping within 5 seconds of administering said fine mist into said nasal cavities or over said mucous membranes;
said fine mist being having average droplet sizes from 0.4-100 microns for enabling at least 50% of said fine mist to adhere to said nasal cavities or said mucous membranes without dripping within 5 seconds of administering said fine mist into said nasal cavities or over said mucous membranes;
oxygen molecules of said reactive oxygen species (ROS) within said aerosolized fine being in a reactive state having oxidation state of O⁻¹ capable of oxidizing bacteria, enzymes and impurities within said mucous membranes;
said aerosolized fine mist being characterized in that when administered over said mucus membranes, it oxidizes bacteria, enzymes and impurities within said mucous membranes thereby forming miniature clusters of active microscopic oxygen bubbles within said mucous membranes which serve as transportation routes and vehicles to disperse said pharmaceutically acceptable mucosal antimicrobial or therapeutic agent(s) into said mucus membranes in about two minutes.

2. The device for treatment of nasal cavities of claim 1, wherein said fine mist is being having a predetermined positive (+) and/or negative (−) electrical charge for enabling at least 50% of said fine mist to adhere to said nasal cavities or said mucous membranes without dripping within 5 seconds of administering said fine mist into said nasal cavities or over said mucous membranes.

3. The device for treatment of nasal cavities of claim 1, wherein said liquid solution consists of sufficient amount of pharmaceutically acceptable astringent(s), thickening agent(s), mucoadhesive(s), bioadhesive(s), excipient(s) and/or alcohol(s) for enabling at least 50% of said fine mist to adhere to said nasal cavities or said mucous membranes without dripping within 5 seconds of administering said fine mist into said nasal cavities or over said mucous membranes.

4. The device for treatment of nasal cavities of claim 1, wherein said pharmaceutically acceptable mucosal antimicrobial or therapeutic agent(s) is (are) selected from from groups (a)-(l).

5. An aerosolized composition for treatment of nasal cavities and/or mucous membranes comprising:
aerosolized fine mist produced from a liquid solution by using a spray bottle or a nebulizer;
said fine mist being comprised of reactive oxygen species (ROS) consisting essentially of hydrogen peroxide from about 0.1% to 5% w/w, w/v; one or more pharmaceutically acceptable mucosal antimicrobial or therapeutic agent(s) from about 0.01% to 5% w/w, w/v; and water;
said liquid solution being having viscosity from 0.8-10,000 cps for enabling at least 50% of said fine mist to adhere to said nasal cavities or said mucous membranes without dripping within 5 seconds of administering said fine mist into said nasal cavities or over said mucous membranes;
said fine mist being having average droplet sizes from 0.4-100 microns for enabling at least 50% of said fine mist to adhere to said nasal cavities or said mucous membranes without dripping within 5 seconds of administering said fine mist into said nasal cavities or over said mucous membranes;
oxygen molecules of said reactive oxygen species (ROS) within said aerosolized fine being in a reactive state having oxidation state of O⁻¹ capable of oxidizing bacteria, enzymes and impurities within said mucous membranes;
said aerosolized composition being characterized in that when administered over said mucus membranes, it oxidizes bacteria, enzymes and impurities within said mucous membranes thereby forming miniature clusters of active microscopic oxygen bubbles within said mucous membranes which serve as transportation routes and vehicles to disperse said pharmaceutically acceptable mucosal antimicrobial or therapeutic agent(s) into said mucus membranes in about two minutes.

6. The aerosolized composition of claim 5, wherein said fine mist is being having a predetermined positive (+) and/or negative (−) electrical charge for enabling at least 50% of said fine mist to adhere to said nasal cavities or said mucous membranes without dripping within 5 seconds of administering said fine mist into said nasal cavities or over said mucous membranes.

7. The aerosolized composition of claim 5, wherein said liquid solution consists of sufficient amount of pharmaceutically acceptable astringent(s), thickening agent(s), mucoadhesive(s), bioadhesive(s), excipient(s) and/or alcohol(s) for enabling at least 50% of said fine mist to adhere to said nasal cavities or said mucous membranes without dripping within 5 seconds of administering said fine mist into said nasal cavities or over said mucous membranes.

8. The aerosolized composition of claim 5, wherein said pharmaceutically acceptable mucosal antimicrobial or therapeutic agent(s) is (are) selected from from groups (a)-(l).

9. A method of treatment of nasal cavities and/or mucous membranes, consisting essentially of:
administering an aerosolized composition into the nasal cavities and/or over mucous membrane of a subject requiring treatment;
said aerosolized composition being produced from a liquid solution by using a spray bottle or a nebulizer;
said aerosolized composition being comprised of reactive oxygen species (ROS) consisting essentially of hydrogen peroxide from about 0.1% to 5% w/w, w/v; sufficient amount one or more pharmaceutically acceptable mucosal antimicrobial or therapeutic agent(s); and water;
said liquid solution being having viscosity from 0.8-10,000 cps for enabling at least 50% of said fine mist to adhere to said nasal cavities or said mucous membranes without dripping within 5 seconds of administering said fine mist into said nasal cavities or over said mucous membranes;
said aerosolized composition being in the form of fine mist having average droplet sizes from 0.4-100 microns for enabling at least 50% of said fine mist to adhere to said nasal cavities or said mucous membranes without dripping within 5 seconds of administering said fine mist into said nasal cavities or over said mucous membranes;
oxygen molecules of said reactive oxygen species (ROS) within said aerosolized fine being in a reactive state having oxidation state of O⁻¹ capable of oxidizing bacteria, enzymes and impurities within said mucous membranes;
said aerosolized composition being characterized in that when administered over said mucus membranes, it oxidizes bacteria, enzymes and impurities within said mucous membranes thereby forming miniature clusters of active microscopic oxygen bubbles within said mucous membranes which serve as transportation routes and vehicles to disperse said pharmaceutically acceptable mucosal antimicrobial or therapeutic agent(s) into said mucus membranes in about two minutes.

10. The method of treatment of claim 9, wherein said fine mist is being having a predetermined positive (+) and/or negative (−) electrical charge for enabling at least 50% of said fine mist to adhere to said nasal cavities or said mucous membranes without dripping within 5 seconds of administering said fine mist into said nasal cavities or over said mucous membranes.

11. The method of treatment of claim 9, wherein said liquid solution consists of sufficient amount of pharmaceutically acceptable astringent(s), thickening agent(s), mucoadhesive(s), bioadhesive(s), excipient(s) and/or alcohol(s) for enabling at least 50% of said fine mist to adhere to said nasal cavities or said mucous membranes without dripping within 5 seconds of administering said fine mist into said nasal cavities or over said mucous membranes.

12. The method of treatment of claim 9, wherein said pharmaceutically acceptable mucosal antimicrobial or therapeutic agent(s) is (are) selected from from groups (a)-(l).

13. A nasal spray or throat spray device, comprising:
a nasal spray or throat spray bottle configured to produce fine mist spray from a liquid solution;
said liquid solution being consisting essentially of hydrogen peroxide from about 0.1% to 5% w/w, w/v;
baking soda and/or sodium chloride from about 0.1% to 5% w/w, w/v; and water;
said fine mist being characterized in that when administered over said mucus membranes, it oxidizes bacteria, enzymes and impurities within said mucous membranes thereby forming miniature clusters of active microscopic oxygen bubbles within said mucous membranes which serve as transportation routes and vehicles to disperse said pharmaceutically acceptable mucosal antimicrobial or therapeutic agent(s) into said mucus membranes in about two minutes.

14. The nasal spray or throat spray device of claim 13 wherein said liquid solution is further comprised of an aromatic compound.

15. The nasal spray or throat spray device of claim 13 wherein said liquid solution is further comprised of alcohol, glycerin and/or sugar alcohol.

16. The nasal spray or throat spray device of claim 13 wherein said liquid solution is further comprised a surfactant and/or a thickening agent.

17. The nasal spray or throat spray device of claim 13 wherein said liquid solution is further comprised a stabilizer and/or a buffering agent.

18. A method of cleansing, disinfection and treatment of nasal cavities comprising:
administering aerosolized fine mist produced from a liquid solution by using a nasal spray bottle into the nasal cavities of a subject by using requiring treatment;
said liquid solution being comprised of reactive oxygen species (ROS) having oxidation state of oxygen as (O⁻¹) from about 0.1-5% w/w, w/v, v/v; and sufficient amount one or more oxygen-containing pharmaceutically acceptable mucosal antimicrobial or therapeutic agent(s) having oxidation state of oxygen as (O⁻²); and water;
said fine mist being characterized in that when administered over said mucus membranes, it oxidizes bacteria, enzymes and impurities within said mucous membranes thereby forming miniature clusters of active microscopic oxygen bubbles within said mucous membranes which serve as transportation routes and vehicles to disperse said pharmaceutically acceptable mucosal antimicrobial or therapeutic agent(s) into said mucus membranes in about two minutes.

19. The method of cleansing, disinfection and treatment of nasal cavities of claim 18 wherein said liquid solution further comprises one or more constituents from groups (a)-(l).

1-9. (canceled)

10. A nasal spray system for dispersing one or more aerosolized therapeutic compound(s) into a nasal mucosa, comprising:
a nasal spray device equipped with a metered pump configured to deliver aerosolized droplets sized 100 microns or less produced from a pharmaceutical composition having a predetermined viscosity and comprising:
hydrogen peroxide about 0.1%-5% w/w;
one or more skin moisturizer(s) about 0.1%-30% w/w;
at least one aromatic compound; and
water,
characterized in that said aerosolized droplets, when administered into nasal cavities, form a substantially self-adhering reactive coating on at least a portion of said nasal mucosa less than 0.5 millimeter thick, thereby enabling oxidation of at least a portion of the mucus of said nasal mucosa in less than 3 minutes, enabling loosening of said at least portion of the mucus for removal from said nasal cavities and simultaneously enabling dispersion of at least a portion of said skin moisturizer(s), said aromatic compound(s) and said water into said nasal mucosa of said nasal cavities by an energy of redox reaction between said hydrogen peroxide and said at least portion of the mucus.

11. (canceled)

12. (canceled)

13. The nasal spray system according to claim 10, wherein said pharmaceutical composition further comprises at least one of the following: pharmaceutically acceptable heterocyclic compound(s) and heterocyclic derivative(s).

14. (canceled)

15. The nasal spray system according to claim 10, wherein said pharmaceutical composition further comprises at least one of the following: chlorhexidine gluconate (CHG) about. 0.01%-0.1% w/w, cetylpyridinium chloride about 0.01%-0.1% w/w, a surfactant, and an astringent.

16. The nasal spray system according to claim 10, wherein said pharmaceutical composition further comprises at least one of the following: borax, boric acid, magnesium sulfate, alum, and a pharmaceutically acceptable salt of sodium.

17. The nasal spray system according to claim 10, wherein said nasal spray device is part of a retail package, the retail package comprising said nasal spray device and a bottle containing said pharmaceutical composition.

18. A method of dispersing one or more aerosolized therapeutic compound(s) into a mucosa, the method consisting essentially of:
administering a spray of aerosolized droplets less than 100 microns in sizes over a mucosa of a subject by using a directional spray device configured to deliver a pharmaceutical composition having a predetermined viscosity and consisting essentially of:
hydrogen peroxide about 0.1%-5% w/w;
one or more skin moisturizer(s) about 0.1%-30% w/w;
one or more of the following: pharmaceutically acceptable heterocyclic compound(s) and heterocyclic derivative(s) about 0.005%-2.5% w/w; and
water,
characterized in that said aerosolized droplets, when administered over said mucosa, form a substantially self-adhering reactive coating on at least a portion of said mucosa less than 0.5 millimeter thick and gradually oxidize bodily fluids of said mucosa in less than 3 minutes, thereby loosening said bodily fluids from said mucosa and simultaneously delivering at least a portion of said skin moisturizer(s), said one or more heterocyclic compound(s), said heterocyclic derivative(s) and said water into said mucosa by an energy of redox reaction between said hydrogen peroxide and said bodily fluids.

19. (canceled)

20. (canceled)

21. The method of dispersing one or more aerosolized therapeutic compound(s) into a mucosa of claim 18, wherein said pharmaceutical composition further consists essentially of at least one of the following: chlorhexidine gluconate (CHG), cetylpyridinium chloride, a surfactant, and an astringent.

22. The method of dispersing one or more aerosolized therapeutic compound(s) into a mucosa of claim 18, wherein said pharmaceutical composition further consists essentially of at least one of the following: borax, magnesium sulfate, alum, and a salt of sodium.

23. The method of dispersing one or more aerosolized therapeutic compound(s) into a mucosa of claim 22, wherein said pharmaceutical composition further consists essentially of at least one of the following: alcohol, from about 5%-70% v/v, chlorhexidine gluconate (CHG), cetylpyridinium chloride, a surfactant, and an astringent.
wherein said skin moisturizer is at least one of the following: glycerin, a derivative of glycerin, aloe vera gel, a polymer, a copolymer, sodium lactate, urea, and a derivative of urea.

24. The method of dispersing one or more aerosolized therapeutic compound(s) into a mucosa of claims 18-23, wherein said directional spray device is part of a retail package, the retail package consisting essentially of said directional spray device and a bottle containing said pharmaceutically acceptable compound.

25. (canceled)

26. (canceled)

27. (canceled)

28. (canceled)

29. A method of dispersing one or more aerosolized therapeutic compound(s) into a nasal mucosa, the method comprising:
configuring a nasal spray device equipped with a metered pump to deliver aerosolized droplets sized 100 microns or less from a pharmaceutical composition;
said pharmaceutical composition being comprised of:
hydrogen peroxide about 0.1%-5% w/w;
one or more skin moisturizer(s) about 0.1%-30% w/w;
one or more of the following: pharmaceutically acceptable heterocyclic compound(s) and heterocyclic derivative(s) about 0.005%-2.5% w/w; and
water, administering said aerosolized droplets into nasal cavities;
characterized in that said aerosolized droplets, when administered into said nasal cavities, form a substantially self-adhering reactive coating on at least a portion of said nasal mucosa less than 5 millimeter thick and gradually oxidize at least a portion of a mucus of said nasal mucosa in less than 3 minutes, thereby loosening said at least portion of the mucus from said nasal mucosa of said nasal cavities for removal and simultaneously deliver at least a portion of said skin moisturizer(s), said heterocyclic compound(s), said heterocyclic derivative(s), and said water into said nasal mucosa by an energy of redox reaction between said hydrogen peroxide and said mucus.

30. (canceled)

31. (canceled)

32. (canceled)

33. (canceled)

34. A system of drug delivery for dispersing one or more aerosolized therapeutic compound(s) into a mucosa, comprising:
a directional spray device equipped with a metered pump configured to deliver aerosolized droplets sized 100 microns or less produced from a pharmaceutical composition having a predetermined viscosity and comprising:
hydrogen peroxide about 0.1%-5% w/w;
sufficient amount(s) of one or more of the following: aromatic compound(s), pharmaceutically acceptable heterocyclic compound(s), heterocyclic derivative(s), and pharmaceutically acceptable compound(s) comprising substituted heterocyclic group(s);
one or more pharmaceutically acceptable surfactant(s); and
water,
wherein said aerosolized droplets, when administered over a mucosa, form a substantially self-adhering reactive coating on at least a portion of said mucosa less than 0.5 millimeter thick and oxidize at least a portion of bodily fluids of said mucosa in less than 3 minutes, thereby loosening said at least portion of the bodily fluids from said mucosa and simultaneously deliver at least a portion of said aromatic compound(s), said heterocyclic compound(s), said heterocyclic derivatives, said pharmaceutically acceptable compound(s) comprising substituted heterocyclic group(s), said surfactant(s) and said water into said mucosa by an energy of redox reaction between said hydrogen peroxide and said bodily fluids.

35. (canceled)

36. (canceled)

37. (canceled)

38. (canceled)

39. The nasal spray system of claims 10, wherein said metered pump is configured to deliver a predetermined amount of said hydrogen peroxide per pump.

40. The nasal spray system of claims 10, wherein said metered pump is configured to deliver about 0.05 ml-0.2 ml per pump of said liquid composition for enabling one more dosage(s) of predetermined amount of said hydrogen peroxide.

41. The method of dispersing one or more aerosolized therapeutic compound(s) into a mucosa of claim 18, wherein said metered pump is configured to deliver a predetermined amount of said hydrogen peroxide per pump.

42. The method of dispersing one or more aerosolized therapeutic compound(s) into a mucosa of claim 18, wherein said metered pump is configured to deliver about 0.05 ml- 0.2 ml per pump of said liquid composition for enabling one more dosage(s) of predetermined amount of said hydrogen peroxide.

43. The method of dispersing one or more aerosolized therapeutic compound(s) into a nasal mucosa of claim 29, wherein said metered pump is configured to deliver a predetermined amount of said hydrogen peroxide per pump.

44. The method of dispersing one or more aerosolized therapeutic compound(s) into a nasal mucosa of claim 29, wherein said metered pump is configured to deliver about 0.05 ml-0.2 ml per pump of said liquid composition for enabling one more dosage(s) of predetermined amount of said hydrogen peroxide.

45. The system of drug delivery for dispersing one or more aerosolized therapeutic compound(s) into a mucosa of claim 34, wherein said metered pump is configured to deliver a predetermined amount of said hydrogen peroxide per pump.

46. The system of drug delivery for dispersing one or more aerosolized therapeutic compound(s) into a mucosa of claim 34, wherein said metered pump is configured to deliver about. 0.05 ml-0.2 ml per pump of said liquid composition for enabling one more dosage(s) of predetermined amount of said hydrogen peroxide.
